(19) United States
(12) Patent Application Publication
(10) Pub. No.: US 2013/0053685 A1
(43) Pub. Date: Feb. 28, 2013

(54) MAGNETIC RESONANCE SYSTEM AND PROGRAM

(71) Applicant: Naoyuki Takei, Tokyo (JP)

(72) Inventor: Naoyuki Takei, Tokyo (JP)

(21) Appl. No.: 13/598,999

(22) Filed: Aug. 30, 2012

(30) Foreign Application Priority Data
Aug. 31, 2011 (JP) 2011-188999

(51) Int. Cl. A61B 5/024; A61B 5/08; A61B 5/055

(52) U.S. Cl. 600/419

(57) ABSTRACT

A magnetic resonance system is provided. The magnetic resonance system includes a unit configured to acquire magnetic resonance signals from a region including liquid in a subject using a first sequence having a flow compensation gradient field for changing a phase of a spin according to a flow rate, and acquire magnetic resonance signals from the region using a second sequence free of the flow compensation gradient field. The magnetic resonance system further includes a biological signal generating unit configured to generate biological signals of the subject, based on the magnetic resonance signals acquired by the first sequence and the magnetic resonance signals acquired by the second sequence.

### CROSS REFERENCE TO RELATED APPLICATIONS

This application claims the benefit of Japanese Patent Application No. 2011-188999 filed Aug. 31, 2011, which is hereby incorporated by reference in its entirety.

### BACKGROUND OF THE INVENTION

The present invention relates to a magnetic resonance system for imaging based on biological signals of a subject, and a program therefor.

As a method for imaging or capturing a blood flow of a subject, there has been known a heartbeat synchronization method for imaging in sync with a heartbeat signal of the subject. See for example, Japanese Unexamined Patent Publication No. 2011-147561.

When imaging is done by the heartbeat synchronization method, an operator needs to mount a heartbeat sensor to a subject. A problem therefore arises in that a working load is placed on the operator. When the imaging is performed using a respiratory synchronization method together, a bellows may be used to acquire a respiration signal. Since the operator needs to mount the bellows to the subject in this case, a working load on the operator further increases. There has therefore been a demand for acquiring a heartbeat signal and a respiration signal even without using a heartbeat sensor and a bellows.

### BRIEF DESCRIPTION OF THE INVENTION

In a first aspect, a magnetic resonance system is provided. The magnetic resonance system includes a unit for acquiring magnetic resonance signals from a region including liquid in a subject using a first sequence having a flow compensation gradient field for changing a phase of a spin according to a flow rate, and acquiring magnetic resonance signals from the region using a second sequence free of the flow compensation gradient field, and a biological signal generating unit for generating biological signals of the subject, based on the magnetic resonance signals acquired by the first sequence and the magnetic resonance signals acquired by the second sequence.

In a second aspect, a program suitable for a magnetic resonance system including a unit for acquiring magnetic resonance signals from a region including liquid in a subject using a first sequence having a flow compensation gradient field for changing a phase of a spin according to a flow rate, and acquiring magnetic resonance signals from the region using a second sequence free of the flow compensation gradient field is provided. The program causes a computer to execute a biological signal generating process for generating biological signals of the subject, based on the signals acquired by the first sequence and the signals acquired by the second sequence.

By using the first sequence having the flow compensation gradient field and the second sequence free of the flow compensation gradient field, biological signals such as a heartbeat signal, a respiration signal, etc. can be acquired even without using a heartbeat sensor and a bellows.

### BRIEF DESCRIPTION OF THE DRAWINGS

FIG. 1 is a schematic diagram of an exemplary magnetic resonance system according to one embodiment.

FIG. 2 is a diagram showing navigator sequences NAV1 and NAV2 used to acquire a heartbeat signal and a respiration signal in the present embodiment.

FIG. 3 is a diagram illustrating a navigator region R.

FIGS. 4A and 4B are diagrams showing echo signals acquired by the navigator sequences NAV1 and NAV2 in parts as the diastole and systole.

FIGS. 5A-5E are explanatory diagrams of experimental results.

FIGS. 6A and 6B are diagrams showing the manner in which a subject breathes in.

FIGS. 7A-7C are explanatory diagrams of comparison results.

FIG. 8 is a diagram showing a sequence chart executed when the liver of the subject is imaged, and a heartbeat signal W2′ and a respiration signal W1′ acquired by navigator sequences NAV1 and NAV2.

### DETAILED DESCRIPTION OF THE INVENTION

Exemplary embodiments will be explained below, but the present invention is not limited to the embodiments specifically discussed herein.

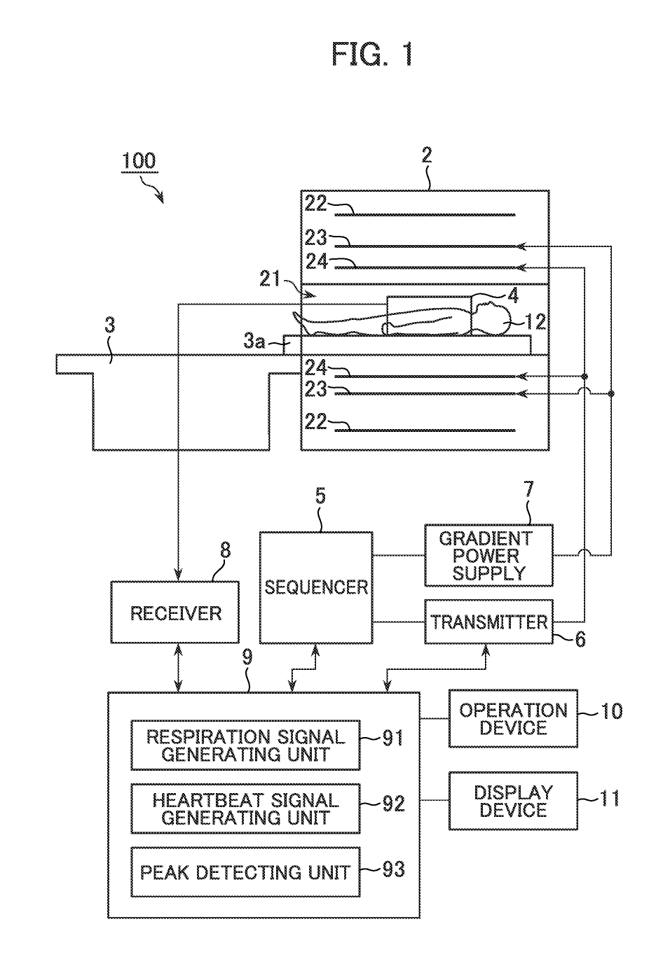

FIG. 1 is a schematic diagram of an exemplary magnetic resonance system according to one embodiment.

The magnetic resonance system (hereinafter called “MR system” where, MR means Magnetic Resonance) 100 has a magnet 2, a table 3, a receiver coil 4 and so on.

The magnet 2 has a bore 21 in which a subject 12 is accommodated, a superconductive coil 22, a gradient coil 23, and a transmitter coil 24. The superconductive coil 22 applies a static magnetic field, the gradient coil 23 applies a gradient pulse, and the transmitter coil 24 transmits an RF pulse. Incidentally, a permanent magnet may be used instead of the superconductive coil 22.

The table 3 has a cradle 3a. The cradle 3a is configured so as to be movable to within the bore 21. The subject 12 is conveyed to the bore 21 by the cradle 3a.

The receiver coil 4 is attached from the chest region of the subject 12 to its abdominal region. The receiver coil 4 receives magnetic resonance signals from the subject 12.

The MR system 100 further has a sequencer 5, a transmitter 6, a gradient power supply 7, a receiver 8, a central processing device 9, an operation device 10 and a display device 11, and the like.

Under the control of the central processing device 9, the sequencer 5 transmits information about a pulse sequence to the transmitter 6 and the gradient power supply 7.

The transmitter 6 outputs a drive signal for driving the RF coil 24, based on the information transmitted from the sequencer 5.

The gradient power supply 7 outputs a drive signal for driving the gradient coil 23, based on the information sent from the sequencer 5.

The receiver 8 signal-processes each magnetic resonance signal received by the receiver coil 4 and transmits the same to the central processing device 9.

The central processing device 9 controls the operations of respective parts of the MR system 100 so as to realize various operations of the MR system 100 such as transmission of information necessary for the sequencer 5 and the display device 11, reconstruction of an image based on data received from the receiver 8, etc. The central processing device 9 is configured by a computer, for example. The central processing unit 9 has a respiration signal generating unit 91, a heartbeat signal generating unit 92 and a peak detecting unit 93, etc.

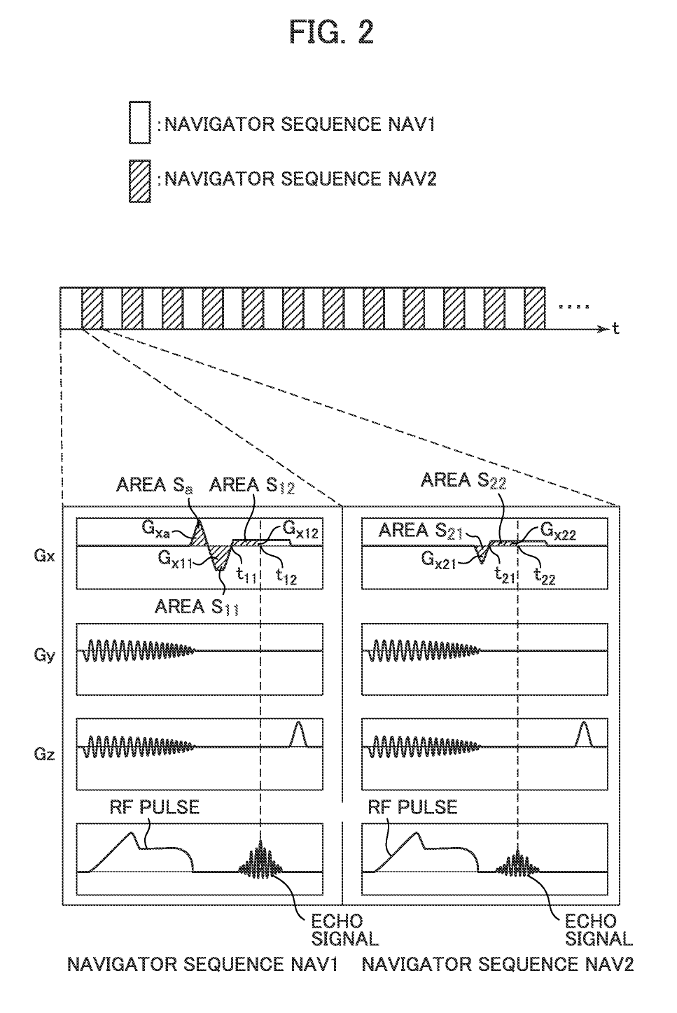

The respiration signal generating unit 91 generates a respiration signal of the subject, based on an echo signal acquired by a navigator sequence NAV1 (refer to FIG. 2) and an echo signal acquired by a navigator sequence NAV2 (refer to FIG. 2).

The heartbeat signal generating unit 92 generates a heartbeat signal of the subject, based on an echo signal acquired by the navigator sequence NAV1 and an echo signal acquired by the navigator sequence NAV2.

The peak detecting unit 93 detects the peak of the respiration signal generated by the respiration signal generating unit 91 and the peak of the heartbeat signal generated by the heartbeat signal generating unit 92.

The central processing unit 9 is one example illustrative of the respiration signal generating unit 91, the heartbeat signal generating unit 92 and the peak detecting unit 93. The central processing unit 9 functions as these units by executing a predetermined program.

The operation device 10 is operated by an operator 13 and inputs various information to the central processing unit 9. The display device 11 displays various information thereon.

The MR system 100 is configured as described above. In the present embodiment, the heartbeat signal of the subject can be acquired even without having to use a heartbeat sensor. Also, the respiration signal of the subject can be acquired even without having to use a respiratory sensor (e.g., bellows). This reason will be explained below while referring to FIGS. 2 through 8.

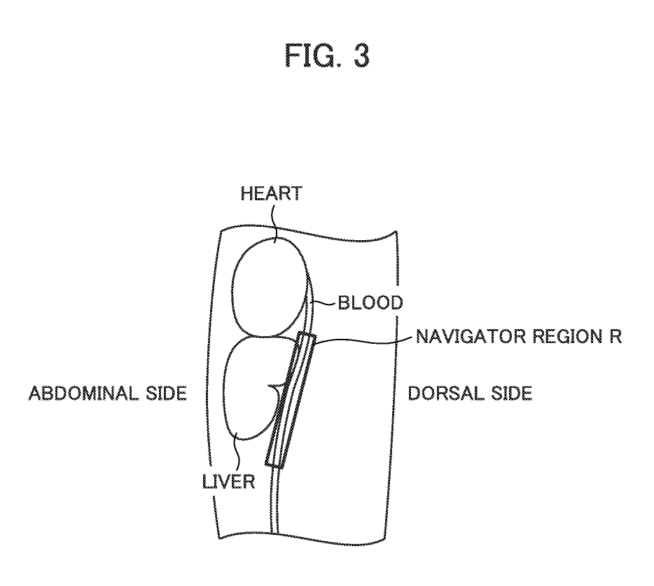

FIG. 2 is a diagram showing the navigator sequences NAV1 and NAV2 used to acquire the heartbeat signal and the respiration signal in the present embodiment, and FIG. 3 is a diagram showing a navigator region R.

The navigator sequences NAV1 and NAV2 are sequences for acquiring or collecting magnetic resonance signals from the navigator region R (refer to FIG. 3) including a blood vessel located in the vicinity of a liver. The navigator sequences NAV1 and NAV2 are executed alternately. The navigator sequences NAV1 and NAV2 will be explained in turn below.

In the navigator sequence NAV1, gradient fields G_{y} and G_{z} inverted alternately in polarity are applied simultaneously with an RF pulse. Consequently, the navigator region R is excited. After the navigator region R has been excited, a gradient field Gₓ for taking out a signal is applied. The gradient field Gₓ has gradient fields Gₓₐ, Gₓ₁₁ and Gₓ₁₂. The gradient field Gₓₐ is a flow compensation gradient field for changing the phase of each spin according to the flow rate. The gradient fields Gₓₐ and Gₓ₁₂ are positive polarities, and the gradient field Gₓ₁₁ is a negative polarity. An area Sₐ of the gradient field Gₓₐ, an area S₁₁ of the gradient field Gₓ₁₁, and an area S₁₂ of the front half portion (between time points t₁₁ and t₁₂) of the gradient field Gₓ₁₂ are set so as to satisfy the following relational expression

Sₐ:S₁₁:S₁₂=1:2:1 (1)

Even in the navigator sequence NAV2, gradient fields G_{y} and G_{z} inverted alternately in polarity are applied simultaneously with an RF pulse in a manner similar to the navigator sequence NAV1. Consequently, the navigator region R is excited. After the navigator region R has been excited, a gradient field Gₓ for taking out a signal is applied. The navigator sequence NAV2 is however different in gradient field Gₓ as compared with the navigator sequence NAV1. In the navigator sequence NAV2, the flow compensation gradient field Gₓₐ is not applied, but only gradient fields Gₓ₂₁ and Gₓ₂₂ are applied. The gradient field Gₓ₂₁ is a negative polarity, and the gradient field Gₓ₂₂ is a positive polarity. An area S₂₁ of the gradient field Gₓ₂₁ and an area S₂₂ of the front half portion (between time points t₂₁ and t₂₂) of the gradient field Gₓ₂₂ are set so as to satisfy the following relational expression:

S₂₁:S₂₂=1:1 (2)

A method for acquiring a heartbeat signal and a respiration signal will next be explained in turn using the navigator sequences NAV1 and NAV2.

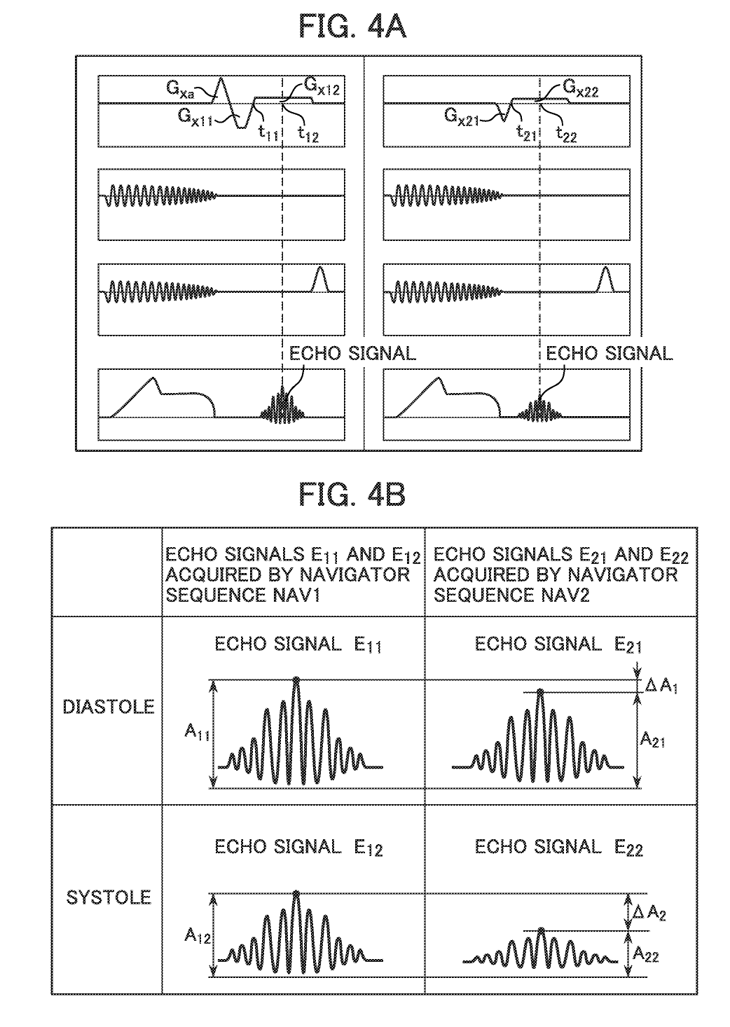

Regarding a method of acquiring the heartbeat signal, FIGS. 4A and 4B are diagrams showing echo signals acquired by the navigator sequences NAV1 and NAV2 in parts as the diastole and systole.

A description will first be made of the echo signals E₁₁ and E₁₂ acquired by the navigator sequence NAV1.

In the navigator sequence NAV1, the area of the gradient field Gₓ is set so as to satisfy the equation (1). With the satisfaction of the equation (1), the phase of each spin in the navigator region R can be brought into approximately the following statuses A1 through A3 according to the flow rate of the spin.

(A1) The phases of stationary spins are rephased at a center time point t₁₂ of a gradient field Gₓ₁₂.

(A2) The phases of spins that perform uniform motion are rephased at the center time point t₁₂ of the gradient field Gₓ₁₂.

(A3) The phases of spins that perform acceleration motion are in the dephasing at the center time point t₁₂ of the gradient field Gₓ₁₂.

During the diastole, the force of the heart pumping the blood becomes weak. Accordingly, the flow rate of the blood flowing through the navigator region R becomes slow. Since the blood can be assumed to remain approximately stationary in this case, the phases in the blood flowing through the navigator region R during the diastole can be assumed to be rephased at the center time point t₁₂ of the gradient field Gₓ₁₂ (status A1). Thus, the amplitude A₁₁ of an echo signal E₁₁ acquired by the navigator sequence NAV1 during the diastole becomes large.

On the other hand, the force of the heart pumping the blood becomes strong during the systole. In this case, the blood flowing through the navigator region R can mainly be separated into the blood that moves at a uniform velocity and the blood that performs acceleration motion. The phases in the blood in uniform motion are rephased at the center time point t₁₂ of the gradient filed Gₓ₁₂ (status A2), whereas the phases in the blood in acceleration motion are in the dephasing at the center time point t₁₂ of the gradient field Gₓ₁₂ (status A3). Thus, the amplitude A₁₂ of an echo signal E₁₂ acquired by the navigator sequence NAV1 during the systole becomes smaller than the amplitude A₁₁ of the echo signal E₁₁.

A description will next be made of the echo signals E₂₁ and E₂₂ acquired by the navigator sequence NAV2.

In the navigator sequence NAV2, the flow compensation gradient field Gₓₐ is not applied, and the area of the gradient field Gₓ is set so as to satisfy the equation (2). With the satisfaction of the equation (2), the phase of each spin in the navigator region R can be brought into approximately the following statuses B1 through B3 according to the flow rate of the spin.

(B1) The phases of stationary spins are rephased at a center time point t₂₂ of a gradient field Gₓ₂₂.

(B2) The phases of spins that move at a uniform velocity are in the dephasing at the center time point t₂₂ of the gradient field Gₓ₂₂.

(B3) The phases of spins that perform acceleration motion are in the dephasing at the center time point t₁₂ of the gradient field Gₓ₁₂.

During the diastole, the blood flowing through the navigator region R can be assumed to remain approximately stationary. Therefore, the phases in the blood flowing through the navigator region R during the diastole can be assumed to be rephased at the center time point t₂₂ of the gradient field Gₓ₂₂ (status B1). Thus, the amplitude A₂₁ of an echo signal E₂₁ acquired by the navigator sequence NAV2 during the diastole becomes large in a manner similar to the amplitude A₁₁ of the echo signal E₁₁.

On the other hand, during the systole, the blood flowing through the navigator region R can mainly be divided into the blood that moves at a uniform velocity and the blood that performs acceleration motion. Thus, when the navigator sequence NAV2 is executed, the phases in the most of the blood are in the dephasing at the center time point t₂₂ of the gradient field Gₓ₂₂ (statuses B2 and B3). Accordingly, the amplitude A₂₂ of an echo signal E₂₂ acquired by the navigator sequence NAV2 during the systole becomes considerably small.

Thus, during the diastole, the amplitude A₁₁ of the echo signal E₁₁ obtained by the navigator sequence NAV1 becomes a value close to the amplitude A₂₁ of the echo signal E₂₁ obtained by the navigator sequence NAV2. On the other hand, during the systole, the amplitude A₂₂ of the echo signal E₂₂ obtained by the navigator sequence NAV2 becomes considerably smaller than the amplitude A₁₂ of the echo signal E₁₂ obtained by the navigator sequence NAV1. Accordingly, when the difference between the amplitudes of the echo signals acquired by the navigator sequences NAV1 and NAV2 is determined, the difference ΔA₁ in amplitude between the echo signals becomes small during the diastole, but the difference ΔA₂ in amplitude between the echo signals becomes large during the systole. Thus, the difference in amplitude between the echo signals varies depending on the diastole or the systole. Therefore, acquiring the echo signals using navigator sequences NAV1 and NAV2 makes it possible to obtain the a heartbeat signal. Experiments were performed to verify it. Experimental results will be explained below.

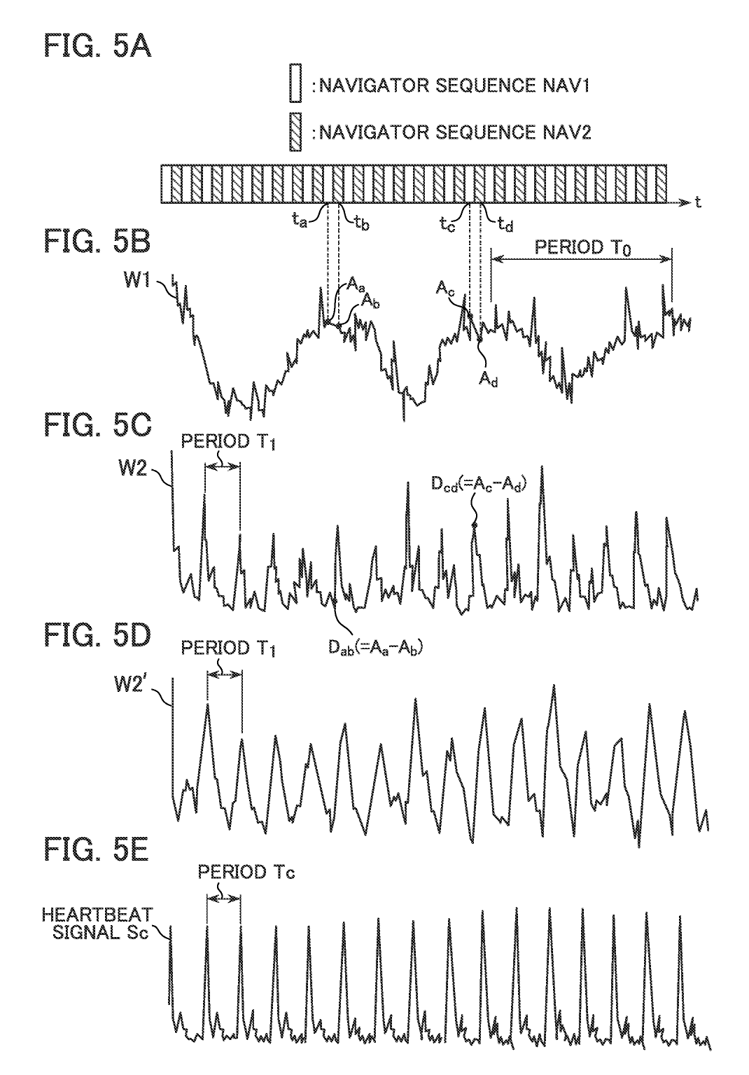

FIGS. 5A-5E are explanatory diagrams of experimental results.

FIG. 5A is an explanatory diagram of sequences used in experiments. In the experiments, the navigator sequences NAV1 and NAV2 shown in FIG. 2 were alternately carried out. The navigator sequences NAV1 and NAV2 are both sequences for acquiring echo signals from the navigator region R (refer to FIG. 3).

FIG. 5B is a diagram that shows a signal W1 representing a temporal change in the amplitude of each echo signal obtained by the sequences of FIG. 5A. Amplitudes Aₐ, A_{b}, A_{c} and A_{d} of four echo signals are concretely shown in the signal W1 of FIG. 5B. The amplitudes Aₐ and A_{b} respectively correspond to the amplitudes of the echo signals acquired by the navigator sequences NAV1 and NAV2 at their times points tₐ and t_{b}. The amplitudes A_{c} and A_{d} respectively correspond to the amplitudes of the echo signals acquired by the navigator sequences NAV1 and NAV2 at their time points t_{c} and t_{d}.

A signal W2 of FIG. 5C indicates a temporal change in the difference between amplitudes adjacent in a time-base direction at the signal W1 of FIG. 5B. Differences D_{ab} and D_{cd} in amplitude are concretely shown in the signal W2 of FIG. 5C as difference's representatives. The difference D_{ab} in the amplitude corresponds to the difference between the amplitudes Aₐ and A_{b}, and the difference D_{cd} in the difference corresponds to the difference between the amplitudes A_{c} and A_{d}. It is understood that when the differences D_{ab} and D_{cd} are compared, a large difference develops between the two. Incidentally, the signal W2 of FIG. 5C had a period T₁ of about 1 second or so.

FIG. 5D is a signal W2′ obtained by removing harmonic components from the signal W2 of FIG. 5C. The signal W2′ of FIG. 5D has a period T₁ of one second or so in a manner similar to the signal W2 of FIG. 5C.

FIG. 5E shows a heartbeat signal SC obtained by a pulse wave sensor. Comparing the signal W2 of FIG. 5C and the heartbeat signal SC of FIG. 5E shows that the period T₁ of the signal W2 approximately coincides with a period T_{c} of the heartbeat signal SC. Accordingly, it is understood that a heartbeat signal including heartbeat information can be obtained by determining the signal W2 representing a temporal change in the difference between the amplitudes. Incidentally, as compared with the heartbeat signal SC obtained using the heartbeat sensor, the signal W2 becomes prominent in noise. Therefore, in the exemplary embodiment, noise is reduced as much as possible. In order to reduce noise, the signal W2′ in which harmonic components have been eliminated may be generated as shown in FIG. 5D. By removing the harmonic components, the signal can be made more approximate to the waveform of the heartbeat signal SC acquired by the heartbeat sensor. As a method for eliminating the harmonic components, a moving average method or the like can be used.

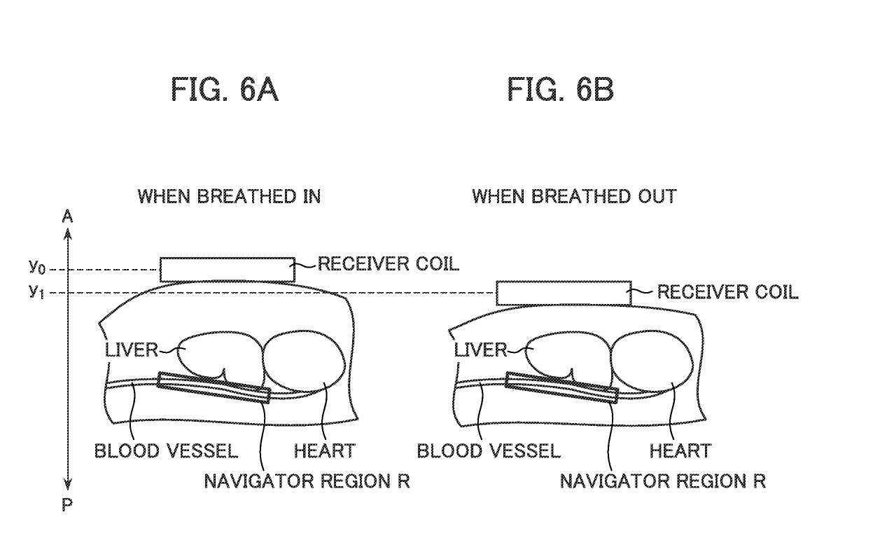

Regarding a method for acquiring a respiration signal, a period T₀ of the signal W1 of FIG. 5B is considered to be indicative of a respiratory period Tᵣ of the subject. This reason will be explained while referring to FIGS. 6A and 6B.

FIG. 6A schematically shows the position of the receiver coil for the subject when the subject has breathed in. Since the abdominal circumference of the subject expands when the subject breathes in, the position of the receiver coil in an AP direction is away from a navigator region R (position y₀).

FIG. 6B schematically shows the position of the receiver coil for the subject when the subject breathes out. Since the abdominal circumference of the subject becomes small when the subject breathes out, the position of the receiver coil in the AP direction becomes close to the navigator region R (position y₁). Thus, with the motion of breathing of the subject, the position of the receiver coil is considered to approach the navigator region R or increase with distance from the navigator region R. Since the received strength of signal becomes large when the receiver coil approaches the navigator region R, the amplitude of an echo signal is considered to be large. On the other hand, since the received strength of signal becomes small when the receiver coil is away from the navigator region R, the amplitude of the echo signal is considered to be small. Thus, the period T₀ of the signal W1 representing the temporal change in the amplitude of each echo signal is considered to be indicative of the respiratory period of the subject. In order to verify this, the respiration signal of the subject was acquired using a bellows and compared with the signal of FIG. 5B.

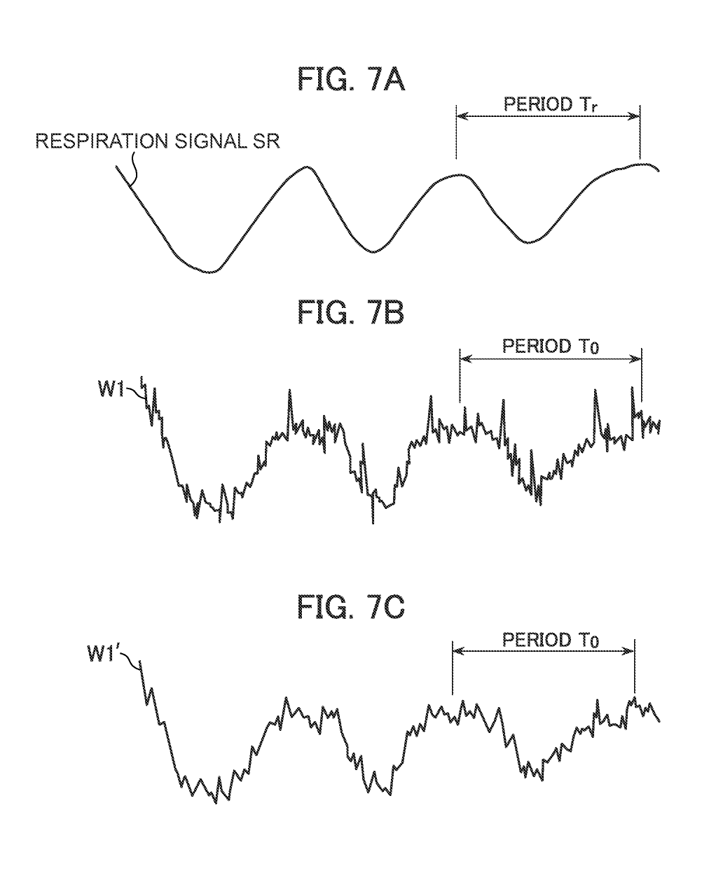

FIGS. 7A-7C are explanatory diagrams of comparison results.

FIG. 7A is a diagram showing a respiration signal SR of the subject, which has been acquired using a bellows, FIG. 7B is a diagram showing the signal W1 of FIG. 5B, and FIG. 7C is a signal W1′ obtained by eliminating harmonic components from the signal W1.

It is understood that when FIG. 7A and FIG. 7B are compared, the period T₀ of the signal W1 in FIG. 7B approximately coincides with the period Tᵣ of the respiration signal SR acquired using the bellows. Accordingly, it is understood that a respiration signal can be obtained by determining the temporal change in the amplitude of each echo signal. Incidentally, as compared with the respiration signal SR obtained by the bellows, the signal W1 becomes prominent in noise. Therefore, in the exemplary embodiment, noise is reduced as much as possible. In order to reduce noise, the signal W1′ in which harmonic components have been eliminated may be generated as shown in FIG. 7C. By removing the harmonic components, the signal can be made more approximate to the waveform of the respiration signal SR acquired by the bellows. As a method for eliminating the harmonic components, a moving average method or the like can be used.

Thus, it is understood that as described above while referring to FIGS. 5A through 7C, the heartbeat signal and the respiration signal can be obtained by the amplitudes of the echo signals based on the navigator sequences NAV1 and NAV2 even without having to use the heartbeat sensor and the bellows.

A description will next be made of one example of a method for imaging the liver of the subject while acquiring the heartbeat signal and the respiration signal using the navigator sequences NAV1 and NAV2 show in FIG. 2.

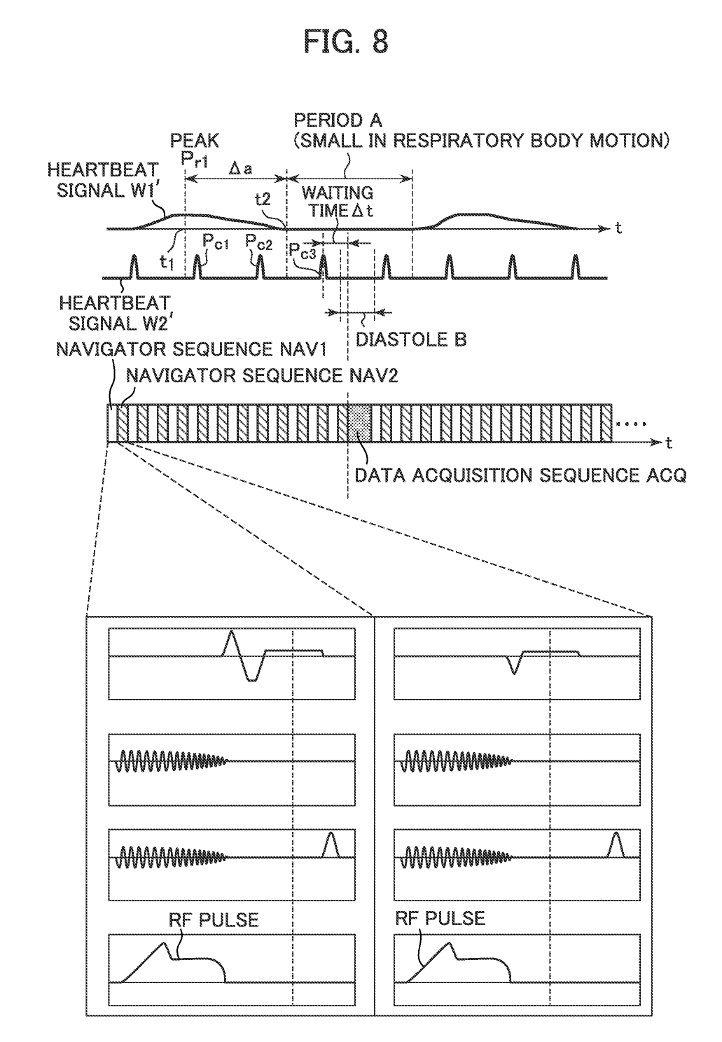

FIG. 8 is a diagram showing a sequence chart executed when the liver of the subject is imaged, and a heartbeat signal W2′ and a respiration signal W1′ acquired by the navigator sequences NAV1 and NAV2. Incidentally, in order to make it easy to view the navigator sequences NAV1 and NAV2, the widths of the navigator sequences NAV1 and NAV2 are broadened out in FIG. 8.

The navigator sequences NAV1 and NAV2 are first alternately executed to acquire echo signals. The respiration signal generating unit 91 (refer to FIG. 1) calculates the amplitude of each of the acquired echo signals and thereby determines a temporal change in the amplitude thereof. Thus, as described while referring to FIGS. 7A-7C, a respiration signal W1 including respiration information of the subject is obtained. Incidentally, as compared with the respiration signal SR (refer to FIG. 7A) acquired by the bellows, the signal W1 becomes prominent in noise. Therefore, the respiration signal generating unit 91 generates a respiration signal W1′ in which harmonic components have been eliminated from the respiration signal W1 in order to reduce noise as much as possible. Eliminating the harmonic components makes it possible to cause the signal to further approach the waveform of the respiration signal SR acquired by the bellows. As a method for eliminating the harmonic components, a moving average method or the like can be utilized.

The heartbeat signal generating unit 92 (refer to FIG. 1) calculates the difference in amplitude between the acquired echo signals and thereby determines a temporal change in the difference in amplitude between the echo signals. Thus, as described while referring to FIGS. 5A-5E, a heartbeat signal W2 including heartbeat information of the subject is obtained. Incidentally, as compared with the heartbeat signal SC (refer to FIG. 5E) acquired by the heartbeat sensor, the signal W2 becomes prominent in noise. Therefore, the heartbeat signal generating unit 92 generates a heartbeat signal W2′ in which harmonic components have been eliminated from the heartbeat signal W2 in order to reduce noise as much as possible. Eliminating the harmonic components makes it possible to cause the signal to further approach the waveform of the heartbeat signal SC acquired by the heartbeat sensor. As a method for eliminating the harmonic components, a moving average method or the like can be utilized.

On the other hand, the peak detecting unit 93 (refer to FIG. 1) detects the peak of the respiration signal W1′ generated by the respiration signal generating unit 91. Since the peak Pᵣ₁ of the respiration signal W1′ appears at a time point t₁ in FIG. 8, the peak detecting unit 93 detects the peak Pᵣ₁. When the peak Pᵣ₁ is detected, the peak detecting unit 93 detects the peak of the heartbeat signal W2′ that appears after the peak Pᵣ₁ of the respiration signal W1′. Then, when the peak detecting unit 93 detects a peak P_{C3} of the heartbeat signal W2′ that appears as a third peak as viewed from the peak Pᵣ₁ of the respiration signal W1′, a data acquisition sequence ACQ for acquiring data about the liver is executed in wait for only a waiting time Δₜ.

The respiratory period of the subject is approximately 4 seconds or so, and the heartbeat period is approximately 1 second or so. Therefore, by executing the data acquisition sequence ACQ in accordance with the above procedure, data about the liver can be acquired during a period A small in body motion based on the respiration of the subject. The waiting time Δₜ is set in such a manner that the data acquisition sequence ACQ is executed during an diastole B. Thus, body motion artifacts caused by respiration can be reduced, and image data in which the blood flow has sufficiently been visualized can be acquired.

Incidentally, in the above description, the data acquisition sequence ACQ is executed in wait for only the waiting time Δₜ when the peak P_{C3} of the heartbeat signal W2′ that appears as the third from the peak Pᵣ₁ of the respiration signal W1′ is detected. The data acquisition sequence ACQ may however be carried out by another method. The data acquisition sequence ACQ may be carried out in wait for a starting time point t₂ of the period A small in respiratory body motion when the peak Pᵣ₁ of the respiration signal W1′ is detected, and in wait for only the waiting time Δₜ when the peak P_{C3} of the heartbeat signal W2′ that first appears after the starting time point t₂ of the period A is detected. As one example of a method for determining whether the starting time point t₂ of the period A is reached, there is known a method for determining in advance a time Δₐ between the time point t₁ of the peak Pᵣ₁ and the starting time point t₂ of the period A (Δₐ=1.5 seconds, for example) and setting a time point where the time Δₐ has elapsed from the peak Pᵣ₁, as the starting time point t₂ of the period A. Incidentally, the respiratory period of the subject is calculated and the value of the time Δₐ may be changed according to the value of the calculated respiratory period.

1. A magnetic resonance system comprising:
a unit configured to:
acquire magnetic resonance signals from a region including liquid in a subject using a first sequence having a flow compensation gradient field for changing a phase of a spin according to a flow rate; and
acquire magnetic resonance signals from the region using a second sequence free of the flow compensation gradient field; and
a biological signal generating unit configured to generate biological signals of the subject, based on the magnetic resonance signals acquired by the first sequence and the magnetic resonance signals acquired by the second sequence.

2. The magnetic resonance system according to claim 1, wherein the liquid is blood and the biological signal is a heartbeat signal.

3. The magnetic resonance system according to claim 2, wherein the biological signal generating unit is further configured to:
determine a first amplitude of each of the magnetic resonance signals acquired by the first sequence and a second amplitude of each of the magnetic resonance signals acquired by the second sequence; and
generate the heartbeat signal, based on a first signal representing a temporal change in the difference between the first amplitude and the second amplitude.

4. The magnetic resonance system according to claim 3, wherein the biological signal generating unit is further configured to eliminate harmonic components from the first signal.

5. The magnetic resonance system according to claim 4, wherein the biological signal generating unit is configured to eliminate the harmonic components using a moving average method.

6. The magnetic resonance system according to claim 1, wherein the liquid is blood and the biological signal is a respiration signal.

7. The magnetic resonance system according to claim 6, wherein the biological signal generating unit is further configured to:
determine a first amplitude of each of the magnetic resonance signals acquired by the first sequence and a second amplitude of each of the magnetic resonance signals acquired by the second sequence; and
generate the respiration signal, based on a second signal representing temporal changes in the first amplitude and the second amplitude.

8. The magnetic resonance system according to claim 7, wherein the biological signal generating unit is further configured to eliminate harmonic components from the second signal.

9. The magnetic resonance system according to claim 8, wherein the biological signal generating unit is configured to eliminate the harmonic components using a moving average method.

10. The magnetic resonance system according to claim 1, wherein the first sequence includes:
the flow compensation gradient field,
a first gradient field having a polarity opposite to the flow compensation gradient field, and
a second gradient field having the same polarity as the flow compensation gradient field, wherein the area of the flow compensation gradient field, the area of the first gradient field and the area of the second gradient field are in a ratio of 1:2:1.

11. The magnetic resonance system according to claim 1, wherein the second sequence includes:
a third gradient field having a polarity opposite to the flow compensation gradient field, and
a fourth gradient field having the same polarity as the flow compensation gradient field, wherein the area of the third gradient field and the area of the front half portion of the fourth gradient field are in a ratio of 1:1.

12. A program suitable for a magnetic resonance system including a unit configured to acquire magnetic resonance signals from a region including liquid in a subject using a first sequence having a flow compensation gradient field for changing a phase of a spin according to a flow rate, and configured to acquire magnetic resonance signals from the region using a second sequence free of the flow compensation gradient field, said program configured to cause a computer to:
execute a biological signal generating process for generating biological signals of the subject, based on the signals acquired by the first sequence and the signals acquired by the second sequence.

13. The program according to claim 12, wherein the liquid is blood and the biological signal is a heartbeat signal.

14. The program according to claim 12, wherein the liquid is blood and the biological signal is a respiration signal.

15. A method for acquiring biological signals of a subject using a magnetic resonance system, said method comprising:
acquiring magnetic resonance signals from a region including liquid in the subject using a first sequence having a flow compensation gradient field for changing a phase of a spin according to a flow rate;
acquiring magnetic resonance signals from the region using a second sequence free of the flow compensation gradient field; and
generating biological signals of the subject, based on the magnetic resonance signals acquired by the first sequence and the magnetic resonance signals acquired by the second sequence.

16. The method according to claim 15, wherein the liquid is blood and generating biological signals comprises generating a heartbeat signal of the subject.

17. The method according to claim 16, further comprising:
determining a first amplitude of each of the magnetic resonance signals acquired by the first sequence and a second amplitude of each of the magnetic resonance signals acquired by the second sequence; and
generating the heartbeat signal, based on a first signal representing a temporal change in the difference between the first amplitude and the second amplitude.

18. The method according to claim 17, further comprising eliminating harmonic components from the first signal.

19. The method according to claim 18, wherein eliminating harmonic components comprises eliminating harmonic components using a moving average method.

20. The method according to claim 15, wherein the liquid is blood and generating biological signals comprises generating a respiration signal of the subject.
